(19) United States
(12) Patent Application Publication
(10) Pub. No.: US 2004/0106986 A1
(43) Pub. Date: Jun. 3, 2004

(54) Surgical marker and an implant

(75) Inventors: Lars Andersson, Skelleftea (SE); Stefan Sehlstedt, Skelleftea (SE)

Correspondence Address: NIXON & VANDERHYE, PC, 1100 N GLEBE ROAD, 8TH FLOOR, ARLINGTON, VA, 22201-4714, US

(73) Assignee: CID CARDIOVASCULAR INNOVATION DESIGN AB, Skelleftea (SE)

(21) Appl. No.: 10/714,981

(22) Filed: Nov. 18, 2003

Related U.S. Application Data
(63) Continuation of application No. PCT/SE02/00965, filed on May 21, 2002.

(30) Foreign Application Priority Data
May 21, 2001 (SE) 0101790-4

(51) Int. Cl. A61F 2/06

(52) U.S. Cl. 623/1.34; 623/1.22

(57) ABSTRACT

The invention concerns a marker intended to facilitate the location of a determined area of an internal organ during secondary surgical procedures where an earlier surgical procedure has been performed, whereby the said internal organ can be native just as it can be a synthetic or autonomic implant and/or a combination of such organs and in this part the area of intervention can be the transition of an anastomosis, whereby to facilitate the said location the marker (4) comprises a part (5) designed as an implant for securing to the area of intervention during a primary operation to form a tactile barrier or boundary for the surgeon during the secondary operation between the area of intervention and the surrounding tissue. The invention also concerns an implant such as a graft provided with such a marker.

The present invention concerns a marker according to the preamble to claim 1 and an implant according to claim 27.

Congenital conditions, illnesses or injuries can cause the impairment of a person's interior organs, whereby a specific organ may have to be replaced with an implant. Transplantations are performed within a number of surgical fields including orthopaedics, abdominal surgery and heart/vascular surgery.

Probably one of the most common transplantations is performed on patients whose blood vessels are no longer capable to serve their purpose. This can be rectified using reconstructive vascular surgery where autogenous blood vessels are transferred from another less vital location of the patient to a new location to replace the diseased or impaired vessel. Vessel implants of synthetic or non-autogenous tissue can be used on patients to replace diseased or impaired native vessels. Implants of this type can in other words constitute a tubular organ with continuous longitudinal passage, the respective ends of which anastomosed to artery ends for bypassing diseased parts of arteries for the transport of blood.

Irrespective of whether the vascular implant or graft is autogenic, non-autogenic or synthetic type, anastomosis will eventually lead to complications in the form of infection, aneurysm, thrombosis, excessive tissue reaction in the form of intimal hyperplasia and stenosis. Long-term patency or unobstructed passage in the vascular graft is dependent on many factors, among which can be named the surgeon's skill, the patient's physical condition, the quality of the graft and, not least, the rate of flow of the blood through the graft. To this part, it has been shown that a large flow volume and high flow rate contribute to a good patency and thereby a high life expectancy of the graft. It is also essential for good patency that the graft has such properties that it can expand and contract radially and longitudinally like a native healthy vessel to follow the pulsing flow from the heart.

Studies have shown that between 60% and 80% of blood vessel transplants must be replaced or rectified in some way within 5 years and consequently it is known beforehand or during the primary operation that the patient will sooner or later have to undergo trying postoperative surgery to rectify an occluded vessel transplant.

Irrespective of where in the body an operation is performed, a troublesome circumstance is that subsequent surgery is generally much more time-consuming and complicated each time it must be performed, as the tissue involved heals with gradually increasing scar formation. Through increased scar formation, the originally unaffected supple tissue will become more disrupted and not exhibit a natural plane of incision. Furthermore, in certain cases, the surrounding tissue will tend to graft itself to the transplant. The said circumstances make it extremely time-consuming to enter the old scar with surgical instruments. It should herewith be understood that the complicated work of exposure considerably increases the time it takes for the already relatively long operation, thereby increasing the risk for the patient.

The procedure is usually performed with the help of a magnifying lens and often in an uncomfortable working position, which means it is many times physically and mentally exhausting for the surgeon. Any reduction of the time taken for exposure is not only significant from the point of view of the working environment but also and above all for the result of the operation and therefore the recovery of the patient. The risks of a reoperation are normally in relation to the time that is necessary to perform the surgery and in certain cases it is not possible to perform a reoperation due to the patient's poor prognosis of survival. Long operation times are consequently a major strain on the human organism and considerable progress would be made if the surgeon was able to quickly, simply and safely enter the tissue on which earlier surgery had been performed and reach the area for the transplant. To be precise, the surgeon should be given the possibility of quickly entering the tissue surrounding the area or the location where the actual surgery is to be performed and first in this area need to carry out the delicate dissection. Reaching the actual area for the operation faster and simpler will not only save time but it will also allow the surgeon's energy and concentration to be utilised more efficiently for the actual vital surgery.

One object of the present invention is therefore to produce an implantable marker that during postoperative surgery can simplify the location of an internal organ where previous surgery has been performed. The said internal organ can for instance be a transplant or, as described in this non-limiting embodiment, a blood vessel implant or a so-called vascular graft. To be precise, a marker is sought that after being applied during the primary operation can render surgery considerably more efficient during a subsequent operation in the old scar.

Another object with the invention is to produce an implantable marker that during postoperative surgery can be located visually both during the actual surgery and beforehand to facilitate determining the area on the body where the surgery should be performed. Since the marker also makes it possible to locate the transplant inside the body prior to surgery, a team of doctors will be able to plan the operation more efficiently. Not least, this can be of great importance during emergency operations such as accidents or the like, as patients with transplants do not always exhibit a normal anatomy. The risk in this connection is obviously that the surgeon may damage vital transplanted organs.

A third object of the invention is to produce a marker that makes it possible to determine in advance the relative position of an inserted surgical instrument. Amongst other things, this would facilitate the treatment of blood clots when applying dissolver or a supporting stent. Since this would make it considerably simpler to determine the precise location of a catheter, e.g. angiographic catheter, in relation to, for instance, the transition between the graft and the native vessel formed during anastomosis. Not least, it would hereby be possible to place the catheter in such a position that the dissolver can be distributed in a manner providing optimum treatment, whereby larger doses of dissolver can be deposited to defined or determined areas, while the risk of complications in the form of haemorrhaging in other places in the body can be avoided.

A fourth object of the invention is to produce a marker that can be used in combination with electric surgical instruments.

The object of the invention can be achieved by it exhibiting the distinctive features and characteristics specified in claim 1. Other distinctive features and advantages are made evident in the remaining claims.

Biostable and biocompatible polymers suitable for long-term implementation in a human body have been known for a long time. Of these, polyurethane, i.e. reaction products of di-isocyanate, has become increasingly common and is used today among other things for the production of vascular grafts, prosthesis, pacemaker housings, etc. Flexible polyurethane foam can be used for the production of elastomers, flexible and bendable articles as well as relatively hard and stiff products.

The manufacture of foam material based on polyurethane and other polymer systems derived from organic polysiloxanes in industrial applications is also well known. The selected composition and manufacturing techniques impact to a wide extent on the properties of the foam products. These can be varied from soft and flexible foam for comfortable applications to hard and stiff materials of the type used for insulation or more constructive purposes. Density and material strength can also be affected and varied by the composition.

Furthermore, it can be said that grafts and blood vessel implants are usually produced from a porous polymer product with fibrils in the form of fluoroethylene plastic (PTFE). Since the said materials are already well tried within the field of transplantation surgery, they could well be used as the basis for the production of a marker in accordance with the present invention. Furthermore, the production of reinforced blood vessel implants based on PTFE materials is previously known, whereby the said reinforcement can constitute passages of non-expanded PTFE arranged in certain areas.

It is also worth considering that the marker could be manufactured as an element comprising several layers sandwiched with a synthetic fibre weave filling of, for example, polyacrylonitrile fibre filament, and a respective outer layer of porous fluoroethylene plastic (PTFE) with fibrilar structure.

Irrespective of the choice of material and manufacturing method applied, the marker should be so designed that it exhibits properties that in all essentials corresponds with the surrounding body tissue in the area where it is intended to be placed. For example, the marker should exhibit an elasticity and flexibility that is comparable with the tissue surrounding it, while it possesses such strength that it cannot easily be cut or penetrated with a scalpel, for example. To this part, the marker can include a layer with tear-resistant non-metallic fibre of one of the following materials with high molecular weight: aromatic plastic, polyethylene plastic, polyvinyl alcohol or acrylonitrile plastic. Furthermore, the marker should be easy to penetrate with puncture instruments such as angiographic needles and possible to cut with a suitable instrument such as a pair of scissors.

The marker is best applied in the area of the transplant's anastomosis and is anchored to both the native tissue and the transplant in an essentially elastic and compliant manner. Hereby, any additional risk of the inserted marker affecting the properties of the native healthy tissue and the transplant to any degree will be reduced. The marker can be suitably kept in place with elements similar to an elastic bands of porous fluoroethylene plastic (PTFE) with fibrilar structure that are arranged running around the said tissue as well as the transplant joined to it. The said band-like element can be suitably secured to the marker with sutures.

Furthermore, the material of which the marker is made can suitably be chosen so that its interior surface does not graft itself to the organ the marker is surrounding so that an artificial plane of incision is hereby created. This interior surface could suitably be chosen from the following materials: PTFE, silicone plastic or other biodegradable material such as Seprafilm®, hyaluronic acid derivate, etc.

The following describes an embodiment of and implantable marker with references to the attached drawing, where

FIG. 1 shows a perspective view of an implantable marker according to the invention applied to an anastomosis between a vessel transplant and a native vessel,

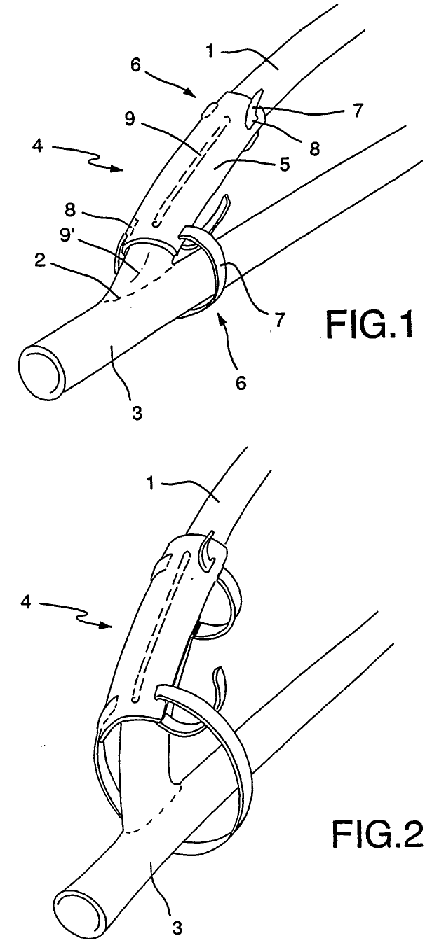

FIG. 2 shows a perspective view of the marker shown in FIG. 1 in a position during application to the area of the anastomosis,

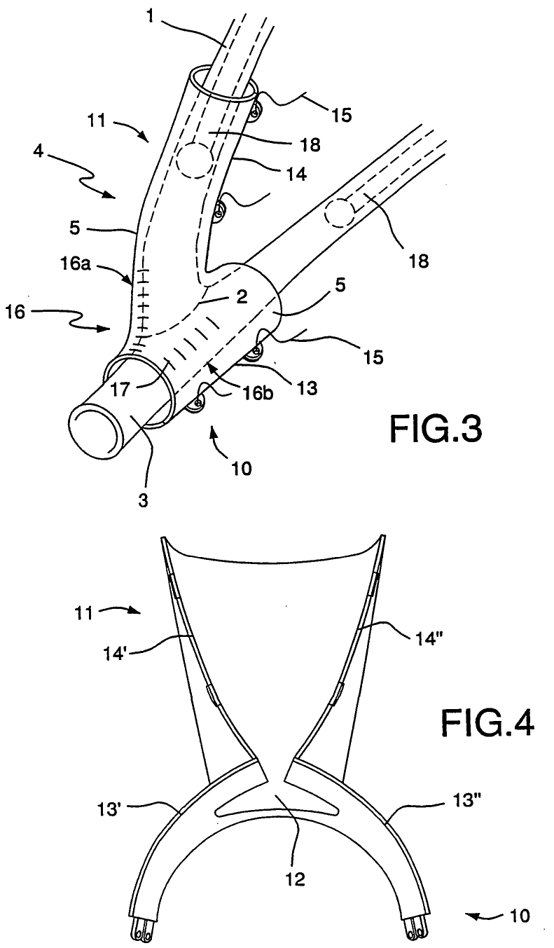

FIG. 3 shows a perspective view of an implantable marker according to the invention in a first alternative embodiment,

FIG. 4 shows the marker in FIG. 3 in an unfolded state,

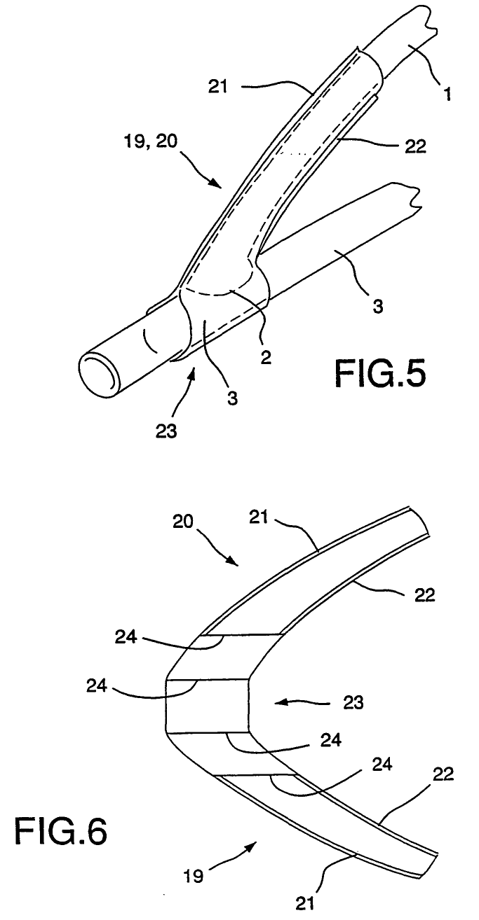

FIG. 5 shows a perspective view of an implantable marker according to the invention in a second alternative embodiment,

FIG. 6 shows the marker in FIG. 5 in an unfolded view,

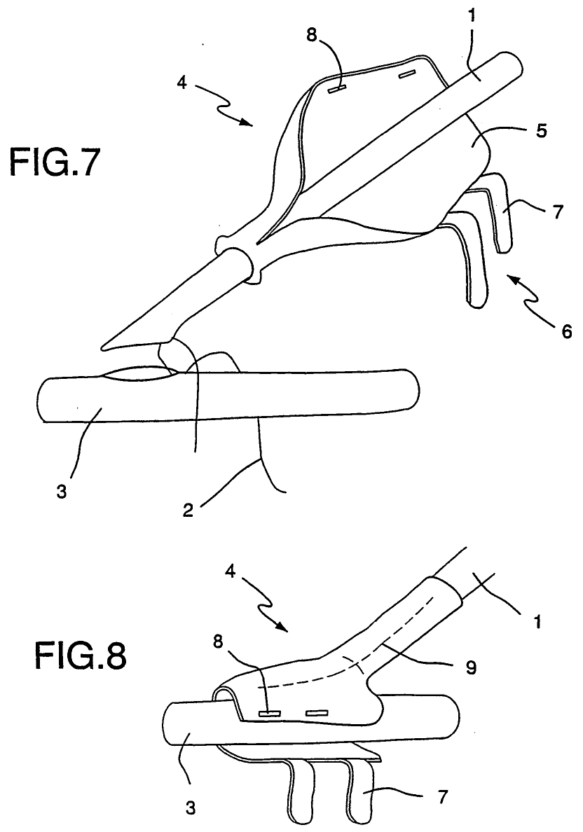

FIG. 7 shows a perspective view of an implantable marker according to the invention in a third alternative embodiment and forming part of a vessel transplant or graft,

FIG. 8 shows the marker in FIG. 7 in position over an anastomosis between the vessel transplant and the native tissue,

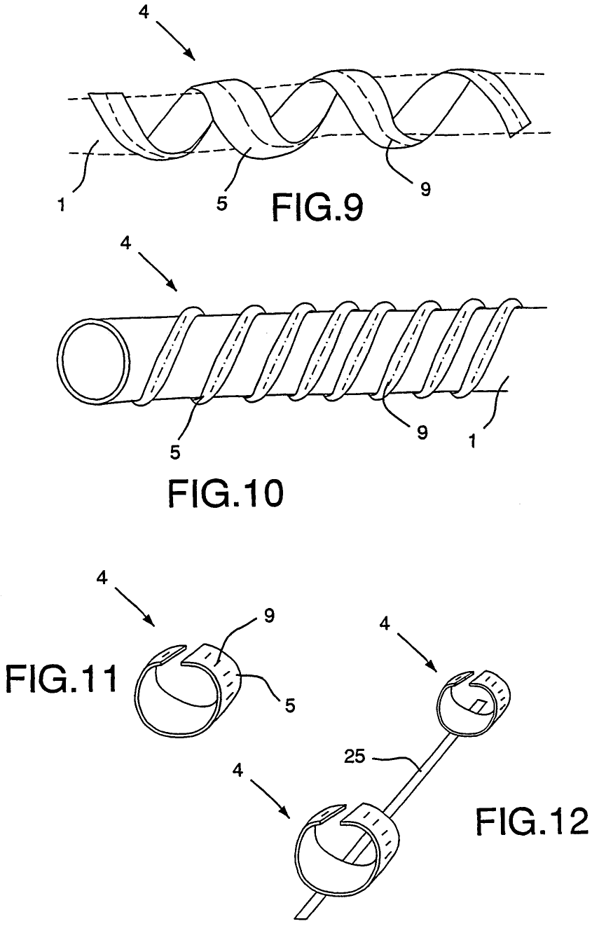

FIG. 9 shows a side view of an implantable marker according to the invention in a third alternative embodiment,

FIG. 10 shows a perspective view of the marker shown in FIG. 9 in an alternative use,

FIG. 11 shows the marker shown in FIG. 10 in an alternative embodiment, and

FIG. 12 shows a perspective view of the embodiment shown in FIG. 11 in a slightly modified embodiment.

In conjunction with a primary operation and transplantation of an organ, which in the described embodiment constitutes a vessel transplant or graft 1 but which, as should be understood, could be any organ, ac graft 1 has been anastomosed to one end of a native artery 3 with sutures 2 to obtain a bypass of the diseased part of the said artery.

An implantable marker according to the invention, generally designated 4, exhibiting the form of an essentially flexible and elastic jacket-like barrier 5 that with fastening elements 6 is anchored to the graft 1 and the native artery 3 in the area where the said parts have been anastomosed in an elastic and compliant manner. Each fastening element 6 constitutes a continuous elastic band 7 which, based on a first side of the barrier and inserted into slotted openings 8 on a second edge of the barrier, are wound around the graft 1 and the native artery 3 as shown in FIGS. 1 and 2. As the band-shaped elements run around the graft 1 and the native artery 3, they can both easily be lifted out and exposed from the surrounding tissue during surgery. To be precise, the entire assembly including the graft 1 and the native artery 3 can be exposed by gripping the marker 4 and lifting it out of the area of incision.

The barrier 5 can be suitably manufactured of a synthetic material that from the point of view of both assembly and manufacturing technique is chosen and designed to give the barrier properties such as elasticity and flexibility that in all essentials are comparable with the properties of the organs to which the marker 4 has been applied. That is to say in this case the graft 1 and the native artery 3 with surrounding tissue. Furthermore, the barrier 5 has at least in some areas been given a high resistance to incision or penetration of the surgeon's scalpel, and is biocompatible and resistant to degradation in the body during a certain period of time. The marker 4 in other words forms a tactile barrier or boundary in the area of the anastomosis between the transplanted organ and the surrounding tissue that is perceptible to the surgeon.

In this respect, it is worth considering that the barrier 5 could be formed from a material sandwiched with a protective weave-like layer of polyacrylonitrile fibre element and a respective outer layer of porous fluoroethylene plastic (PTFE) with fibrilar structure. Alternatively, the barrier 5 could suitably comprise a sandwich layer of tear-resistant fibres of the following materials with high molecular weight: aromatic plastic, polyethylene plastic, polyvinyl alcohol or acrylonitrile plastic. On the said tear-resistant material could be applied a biocompatible material as a covering or coating, the material suitably comprising one of the following materials: fluoroethylene plastic (PTFE) or cellulose nitrate.

The sandwich layer can comprise a scrim of non-metallic fibre such as aromatic fibre, whereby the scrim filling can suitably be run at an angle of between 30° and 60°, primarily 45° to the longitudinal axis of the weave. A sandwich material is hereby obtained with isotropic properties, i.e. with uniform properties in a longitudinal, transverse and diagonal direction. The possibility of the surgeon mistakenly penetrating the material is thereby further encumbered and any fatigue in the material is also counteracted.

The barrier 5 has also been given such a form and extent that when applied to the graft 1 as well as the native artery 3, it at least covers the outside of the said parts in the areas where they have been anastomosed. Active protection of the organ is hereby provided in the area that must be exposed for a reoperation. In order to prevent any negative impact on the vascular graft, the barrier part of the marker is so situated and applied elastically that it is located at a certain distance from the outside of the graft. By designing the barrier with a smooth and non-porous exterior, cell growth and penetration of the graft in which the barrier forms a protective boundary or artificial plane of incision between the surrounding tissue and the graft.

As described above, the barrier 5 protects both the graft 1 and the native artery 3 from penetration of sharp edged instruments, whereby the surgeon can quickly and safely enter the old scar to expose the graft and the artery. The barrier 5 described herein has for this purpose been given a form that in all essentials corresponds with the exterior of the said blood vessel and consequently, the barrier mainly exhibits the form of a fully or partially semicircular casing. By the addition of colouring pigment, the barrier 5 has been given a colour that contrasts it to the surrounding tissue and/or the colour of the surrounding organ. So that the surgeon can be given a simple visual indication as soon as the marker 4 has been located, the colour can suitably be chosen among any of the so-called signal colours normally used for attracting attention. For this part, a suitable colour for the barrier would be yellow or orange, alternatively white.

Since the synthetically produced marker 4 is essentially transparent to X-rays, it has been given a radiographic marker 9 in the form of an X-ray dense metallic wire imbedded in the marker's 4 barrier 5. Through a simple X-ray examination, the precise location of the anastomosis can thereby be established and therefore the location of the incision can also be predetermined in a precise manner. Furthermore, the interventional radiologist can thereby be given a very important guide to where the anastomosis for a specific vessel construction is located.

The radiographic marker 9 imbedded in the marker 4 makes it possible to identify any transplants in a body that are not known to the surgeon before surgical procedure, which, not least during emergency operations such as accidents, can be of great value as patients with transplants do not necessarily exhibit a normal anatomy. In this part, it should be understood that the conventional synthetic graft 1 as shown in FIG. 1 could itself be given a radiographic marker in the form of an X-ray dense metallic wire 9′ imbedded in the outer casing of the graft.

FIGS. 3 and 4 show an alternative embodiment of a marker 4 according to the invention in which is arranged a barrier 5 that principally exhibits the form of a hose to continuously surround and cover both part of the graft 1 as well as the native artery 3 in the area where they are anastomosed. This hose-like barrier 5 can be suitably manufactured of a synthetic material that either is essentially elastic or exhibits only a limited elasticity but otherwise in all essentials exhibits properties that correspond with those described above. The hose-like barrier 5 is hereby designed in one piece and exhibits two tubular parts joined at an angle and generally designated 10 and 11 respectively with an open connection between them via the opening 12. The tubular parts 10, 11 can be separated along the lines 13 and 14 respectively so that the barrier 5 can be opened and mounted over the anastomosed area of the graft 1 and the native artery 3.

The marker 4 is secured to the anastomosed area by its free edges 13′, 13″ and 14′, 14″, which define the separation lines 12, 13 of the tubular parts 10, 11, being joined using the means of attachment 6 in the form of sutures or flexible strips 15 as shown in FIG. 3. If the barrier 5 on the other hand is manufactured in a slightly stiffer material but still with some degree of resilient elasticity, the barrier 5 could in a similar manner be twisted over the said anastomosed area by moving the free edges 12′, 12″ and 13′, 13″ away from each other. A marker 4 placed over the anastomosed area in this way is secured in place with a shaped fastener.

Precisely as described above, this embodiment also incorporates a radiographic marker 16 imbedded in the barrier 5. Unlike previous descriptions herein, this radiographic marker 16 is designed as a first 16a and a second scale 16b extending along the barrier 5 in the direction of the graft and the native artery respectively. The said scales 16a, 16b herewith contain a series of wires 17 located a distance from each other, whereby the scale zero or origin can suitably be situated on the area anastomosed with sutures 2. FIG. 3 illustrates a surgical instrument 18 that could be of the type used to treat blood clots in a known procedure by introducing dissolver or applying a supporting stent inside the blood vessel. With help of X-rays, the path of the instrument 18 in the vessel can be traced while the said scales 15a, 15b make it possible to determine precisely where the instrument is located inside the vessel relative to the anastomosed area and thereby it is possible to determine the optimum position for the instrument.

FIGS. 5 and 6 show the marker 4 in a second alternative embodiment in which its barrier 5 is formed of a hard and rigid material of the kind described above and which can be brought to surround in a circumferential and covering manner the graft 1 and the native artery 3 in the area of their anastomosis. The barrier is split and comprises two jacket-shaped generally designated 19 and 20 respectively pieces, the outer edges 21, 22 of which have means of attachment not shown in detail herein but can suitably comprise interacting snap fasteners so that the said jacket-shaped pieces can be locked closed.

The two jacket-shaped pieces 19, 20 are flexibly joined to each other via a central intermediate piece generally designated 23 so that their edges can be brought together and thereby surround the graft 1 and the native artery 3 as shown in FIG. 5 or separated to an open position as shown in FIG. 6. As should be understood from studying FIGS. 5 and 6 more closely, the central piece 23 is also intended to surround part of the native artery 3 in a compliant manner and at a distance when the jacket-shaped parts are in their closed positions. The central piece 23 is therefore provided with several bending notches 24.

In certain applications of the invention, it ought to be suitable to have the marker according to the invention form a continuous section together with the transplant in order to be applied to the area of intervention as a tactile barrier or boundary once the transplant is in place in the patient. For this reason, the marker 4 is shown in FIGS. 7 and 8 in a third embodiment in which it forms part of an essentially synthetic vascular graft 1. As shown in the figure, the marker's 4 barrier 5 has been given the form of what can be described as a skirt arranged in the area of the end of the graft, which with sutures 2 is intended to be anastomosed to the native blood vessel 3. The said skirt-like barrier has been given such a form and is manufactured in the same manner as described above of a material with such properties that the barrier 5, like a covering or coating, can be twisted over the anastomosed area so that it can surround the anastomosed area as shown in FIGS. 1 and 2. Furthermore, the marker has a radiographic marker 9 and fastening elements 6 in the form of flexible band-shaped means 7, which extending from one side of the barrier can be inserted through slotted openings 8 situated on the other side of the barrier.

FIG. 9 shows the marker 4 according to the invention in a possible alternative embodiment with a barrier 5 that in the main exhibits a spiral shape and being made of one of the aforesaid materials has been given such a degree of flexibility that it can quite simply be wound around a graft or an anastomosed area and can be secured in place thereby. The barrier has furthermore a radiographic marker in the form of a metallic wire 9 imbedded in the said spiral part. FIG. 10 shows the spiral marker 4 in which it is arranged in a circumferential and essentially surrounding manner to an artificial graft 1, in other words in principle as a protective reinforcement against the penetration of surgical instruments, which can be, but not necessarily, imbedded in the covering of e.g. PTFE material that forms the outer covering of the graft. As described above, the said spiral has a radiographic marker in the form of a metallic wire 9.

In FIG. 1 the marker 4 according to the invention has been provided with a barrier 5 in the form of a generally ring or collar-shaped body with an opening defined by two ends a distance from each other. In order for the collar-like marker 4 to be secured to the anastomosed area in an essentially enclosing manner, the body is resilient or elastically compliant so that the opening can be expanded and thereby twisted over the graft or other similar organ. It should be understood, however, that the barrier, as described above, is manufactured of a material with such properties and in such an area-wise and in this part axial extent that it forms a tactile barrier surrounding and thereby protecting the anastomosed area. The barrier 5 has a radiographic marker in the form of a number of metallic wires 9 extending axially, which, imbedded in the material, are distributed evenly at equal distances from each other along the circumference of the barrier.

FIG. 12 shows the collar-shaped marker 4 in an embodiment whereby two uniform markers located a distance from each other are joined together with a relatively heavy intermediate piece 25 extending axially between them.

Practical trials have shown that the present marker is also suitable for use in combination with the type of known electrical instrument that has been increasingly used for surgical procedures recently. This is to say, the type usually named electric surgical instruments where the knife itself is an electrode through which current is conveyed at a high frequency (100 kHz-5 MHz). For this purpose, it has also been shown that designing the marker in such a way that it can act as an insulator offers the advantage of essentially stopping the cutting action of the electric knife as soon as it makes contact with the marker. In this respect it can be said that very good results have been obtained in cases where silicone rubber has been used to provide the marker with its insulating properties. The thickness of the insulating layer is naturally chosen in relation to the expected current strength being used for the procedure but a thickness of approximately 1-2 mm ought to be sufficient in most cases.

In conjunction with a patient undergoing transplantation of a blood vessel, the marker 4 according to the invention is applied and secured as protection in the area of the transplanted vascular graft 1 and the native artery 3 to which the graft has been anastomosed. Ahead of a planned reoperation the patient will be X-rayed, whereby the location in the body of the marker 4 can be established. Once the surgeon has entered the patient's tissue and located and exposed the marker 4, it can be removed from its position in the area between the graft 1 and the native artery. When the procedure is finished, which could include the replacement of the graft 1, the marker 4 of the present type can again be applied to protect the area of the new graft and the native artery to which the graft has been anastomosed.

The present invention is not limited to the above description and as illustrated in the drawings but can be changed and modified in a number of different ways within the framework of the idea of invention specified in the following claims.

1. Marker intended to facilitate the location of a determined area of an internal organ during secondary surgical procedures where an earlier surgical procedure has been performed, whereby the said internal organ can be native just as it can be a synthetic or autonomic implant and/or a combination of such organs, whereby the area of intervention can be the transition of an anastomosis, characterised in that that marker (4) comprises a part (5) designed as an implant for securing to the area of intervention during a primary operation to form a tactile barrier or boundary for the surgeon during the secondary operation between the area of intervention and the surrounding tissue.

2. Marker according to claim 1, whereby the barrier (5) exhibits a contrasting colour to or differs considerably from the colour of the tissue and/or the organ surrounding the area of intervention.

3. Marker according to claim 2, whereby the colour is chosen among any of the colours orange, yellow or white.

4. Marker according to any one of the previous claims 1-3, whereby the barrier (5) is intended to resist the cutting or penetration of surgical instruments such as a scalpel or similar.

5. Marker according to any one of the previous claims 1, whereby the barrier (5) comprises a radiographic marker (9, 16).

6. Marker according to claim 5, whereby the radiographic marker (16) is designed as a graduated scale (16a, 16b) extending in a determined direction along the barrier.

7. Marker according to claim 6, whereby the graduated scale (16a, 16b) comprises a series of wires (17) of a material that is X-ray dense and that in a series after each other are imbedded in the barrier (5) at a determined distance from each other.

8. Marker according to claim 7, whereby the X-ray dense material comprises a metallic material.

9. Marker according to any one of the previous claims, whereby the barrier (5) comprises a biocompatible material that is resistant to degradation for at least a determined period of time and is also inert to bodily tissue and fluids.

10. Marker according to any one of the previous claims, whereby the biocompatible material comprises one of the following materials: fluoroethylene plastic (PTFE) or cellulose nitrate.

11. Marker according to claim 9, whereby the biocompatible material is applied as a coating on the barrier (5).

12. Marker according to the claims, whereby the barrier (5) is flexible and comprises an inner layer of cut-resistant fibre on which the biocompatible material is applied as an outer surrounding layer.

13. Marker according to claim 11, whereby the cut-resistant non-metallic fibre is chosen from the following materials with high molecular weight: aromatic plastic, polyethylene plastic, polyvinyl alcohol or acrylonitrile plastic.

14. Marker according to any one of the previous claims 1-11, whereby the barrier (5) is designed as a hard and rigid jacket.

15. Marker according to any one of the previous claims, whereby the barrier (5) exhibits an exposed inside intended to be applied facing the internal organ (1, 3) and means of attachment (6) for securing the barrier to the said internal organ arranged to extend across the said internal organ.

16. Marker according to claim 15, whereby the means of attachment (6) comprise flexible bands (7).

17. Marker according to claim 12 or 13, whereby the flexible barrier (5) has been given such a form that it can enclose the area of intervention of the internal organ in a continuous circumferential manner and that the butting edges of the surrounding part are intended to be secured to each other as a means of attachment (6).

18. Marker according to claim 17, whereby the means of attachment (6) comprise flexible bands (15) or sutures.

19. Marker according to claim 12 or 13, whereby the flexible barrier (5) comprises tubular parts (10, 11) and exhibits lines (13, 14) along which these parts are separable allowing the barrier to be applied to the internal organ by bending out.

20. Marker according to claim 19, whereby the tubular barrier comprises two tubular parts (10, 11) located at an angle to each other and arranged with an open connection to each other, whereby the separation lines (13, 14) are arranged running along the circumference of the said tubular parts.

21. Marker according to claim 14, whereby the barrier (5) comprises two jacket-shaped pieces (19, 20) that between them are intended to house the internal organ, whereby the said jacket-shaped pieces are provided with means of attachment (21, 22) on their outer edges allowing these to be secured to each other.

22. Marker according to claim 21, whereby the means of attachment (21, 22) comprises interacting snap fasteners.

23. Marker according to claim 14, whereby it comprises two jacket-shaped pieces (20, 21) located on each side of a central piece (23) with which they are flexibly joined for bringing together to surround the internal organ where they are secured to each other with means of attachment (21, 22) acting between the said pieces, whereby the said central piece is also intended to surround a part of the internal organ when the jacket-shaped pieces are in their closed position.

24. Marker according to claim 23, whereby the central piece is provided with joint or bending notches allowing it to be bend compliantly around part of the internal organ.

25. Marker according to any one of the previous claims, whereby the barrier (5) is intended to form an electric insulator or has at least been provided with a degree of insulating properties.

26. Marker according to claim 25, whereby the insulating material comprises silicone rubber.

27. Implant manufactured of synthetic, biologic or semi-synthetic material, characterised in that it comprises a marker (4) of the type specified in claim 1.

28. Implant according to claim 27, whereby it comprises a vascular graft 1.
